(19) United States
(12) Patent Application Publication
(10) Pub. No.: US 2016/0030117 A1
(43) Pub. Date: Feb. 4, 2016

(54) OPERATION OF A MEDICAL ROBOTIC DEVICE

(71) Applicant: Philip Mewes, Nurnberg (DE)

(72) Inventor: Philip Mewes, Nurnberg (DE)

(21) Appl. No.: 14/810,566

(22) Filed: Jul. 28, 2015

(30) Foreign Application Priority Data
Jul. 30, 2014 (DE) 102014214935.5

(51) Int. Cl. A61B 19/00; A61B 17/17; A61B 17/16; A61B 5/00

(52) U.S. Cl. CPC A61B 19/2203; A61B 5/4836; A61B 17/1703; A61B 17/1671; A61B 2019/5287; A61B 2019/5265; A61B 2019/5291; A61B 2019/2207; A61B 2019/5295; A61B 2010/045

(57) ABSTRACT

The embodiments relate to a method for compensating for a deterioration in registration accuracy of a medical robotic device relative to a body to be operated on, the method including selecting at least one landmark on an initial image data record of the body, registering the medical robotic device relative to the body, positioning the end effector in the vicinity of the landmark, recording an intraoperative image data record in which the end effector is captured with a region of the body adjacent to the end effector, determining the position and/or the orientation of the end effector in the intraoperative image data record, comparing this position and/or orientation with the landmark and identifying any divergence, and repositioning the end effector in order to compensate for the divergence, in order thereby to achieve greater precision during the operative intervention.

### CROSS-REFERENCE TO RELATED APPLICATIONS

This application claims the benefit of DE 10 2014 214 935.5, filed on Jul. 30, 2014, which is hereby incorporated by reference in its entirety.

### TECHNICAL FIELD

The embodiments relate to a method for compensating for a deterioration in registration accuracy of a medical robotic device relative to a body to be operated on, the deterioration occurring during an operative intervention.

### BACKGROUND

In the case of operative interventions assisted by medical robotic devices, (e.g., devices that are capable of autonomously performing a movement or autonomously preventing specific movements of the device during the intervention), these devices are registered relative to the body to be operated on. Registration refers to the creation of an unambiguous relationship in a mathematical sense between the positions in a reference system of the medical robotic device and in the reference system of the body to be operated on. This corresponds to a calibration or an alignment relative to the body to be operated on. For example, this may be effected by using a shared system of coordinates for the medical technology device and for image data of the body to be operated on, for example. The medical robotic device has knowledge of where the device or a specific part of the device is positioned relative to the body to be operated on, and how a movement of the medical robotic device affects this position. Different data records may also be registered relative to each other, for example, such that a position in one image data record may be unambiguously assigned to a position in another image data record.

The registration accuracy is therefore a measure of the correspondence of a supposed position to an actual position, for example a supposed position of the medical robotic device relative to the body to be operated on, the supposed position e.g. forming the basis of control instructions for the device, and the actual position of the medical device relative to the body. A high level of registration accuracy is desirable for obvious reasons.

As a result of various influences, the registration accuracy is negatively affected during an operative intervention. For example, changes in the anatomy of the patient due to natural or external interference cause a divergence of the supposed and the actual position of the body to be operated on. Natural changes include, for example, respiration, heartbeat, peristalsis, or physiological changes caused by the flow of blood. External interference that may cause changes in the anatomy and/or the geometry of the body to be operated on includes, for example, a deliberate change in the positioning of the patient or changes produced by the operative intervention itself in the body to be operated on.

Furthermore, inaccuracies of the medical robotic device itself contribute to a deterioration in the registration accuracy. These include, for example, kinematic inaccuracies in global and/or relative positioning accuracy, or inaccuracies in so-called hand-eye calibration of a medical device that interacts with the medical robotic device.

Finally, an inaccurate initial registration between the medical robotic device and the anatomy of the body to be operated on, e.g., an inaccurate initial association between the positions of a medical robotic device and the body to be operated on, is also a possible cause of poor registration accuracy.

These changes and/or inaccuracies present problems in the context of treatment techniques requiring a high degree of spatial precision.

### SUMMARY AND DESCRIPTION

The scope of the present invention is defined solely by the appended claims and is not affected to any degree by the statements within this summary. The present embodiments may obviate one or more of the drawbacks or limitations in the related art.

The object of the present embodiments is to provide a method by which greater precision may be achieved in the context of an operative intervention using a medical robotic device.

A method for compensating for a deterioration in registration accuracy of a medical robotic device relative to a body to be operated on, the deterioration occurring during an operative and/or diagnostic intervention, includes a repositioning of the medical robotic device in a series of acts. In particular, the repositioning may take place automatically in this case. The medical robotic device in this case has an end effector for performing a diagnostic and/or therapeutic measure. In the field of robotics, an end effector may denote a final element in a series of consecutively disposed elements of the medical robotic device, wherein the elements may be moved relative to each other by control instructions. For example, the end effector may be a drilling template that is to be held in a certain position, or a biopsy needle that will be used to take a sample from a specified tissue.

A selection is made of at least one landmark, which is to be reached by the end effector, on an initial image data record of the body to be operated on. The selection may be made by an operator, for example. The landmark may be a position and/or an orientation in which the end effector is oriented at the position. In particular, the landmark may be a three-dimensional vector at a position. A position may be a point of a soft-tissue organ that has been selected for a biopsy, for example. A three-dimensional vector may represent the location of a pedicle screw that is to be inserted, for example. In this context, “reaching a position or landmark” is also understood to signify moving into a known, predefined spatial relationship relative to the position or landmark. Therefore, a position or landmark may also be reached when the end effector is situated at a predefined distance from the position or landmark, particularly with a predefined orientation. The initial image data record may be a preoperative image data record that is recorded, e.g., one month before the intervention, or an intraoperative image data record that is recorded, e.g., directly before or during the intervention and therefore represents the body to be operated on at the time of the operative and/or diagnostic intervention.

A further act includes an initial registration of the medical robotic device relative to the body to be operated on. In particular, this may be effected by the initial image data record if the initial image data record is an intraoperative image data record, or by other intraoperative image data records that are registered with the initial image data record if the initial image data record is not an intraoperative image data record. Such a further intraoperative image data record may be, e.g., a 2D fluoroscopic image that is registered with a 3D volume. The medical robotic device, and in particular its end effector, is therefore moved into a clearly defined and precisely known position and orientation relative to the body to be operated on and/or the landmark.

In a further act, the end effector is positioned in the vicinity of the landmark. This may be performed autonomously by the device or manually by an operator. The vicinity is understood here to signify an area that allows an image data record to be recorded as described in an additional act. In this act, a recording is made of an intraoperative image data record in which the end effector is captured with a region of the body that is adjacent to the end effector, the region including a bodily section that features at least one landmark in the initial image data record. In particular, if a plurality of landmarks are to be reached during the operative and/or diagnostic intervention, and a plurality of landmarks are present in the adjacent region, it may therefore be possible to use the same intraoperative image data record more than once, e.g., for the purpose of positioning the end effector in the vicinity of a plurality of landmarks.

In a further act, the position and/or the orientation of the end effector is determined in the intraoperative image data record. Following thereupon, a comparison is made between this position and/or orientation and the landmark, and any divergence is identified. If applicable, this divergence then represents a measure of a deterioration in registration accuracy. Additionally, the end effector is repositioned in order to compensate for the divergence and hence the deterioration in registration accuracy, such that position and/or orientation of end effector corresponds to the landmark again. The intraoperative imaging is therefore used for the purpose of directly controlling a robotic intervention. This has the advantage that the registration accuracy is improved again and greater precision is achieved during the intervention in respect of the diagnostic and/or therapeutic measure performed using the medical robotic device. As a result of using the intraoperative image data records, which include both the end effector and bodily sections that have a landmark in the initial image data record, it is consequently possible to compensate for both changes that occur in the geometry of the body to be operated on, (e.g., the anatomy of the patient), and are produced by natural or external interference, and kinematic inaccuracies of the medical robotic device itself.

In particular, provision may be made for performing acts a) to g) or acts c) to g) in the sequence listed.

In an advantageous embodiment, provision is made for acts c) to g) to be performed repeatedly during an operative and/or diagnostic intervention. In this case, the act of repositioning the end effector then also corresponds to the act of positioning the end effector in the vicinity of the landmark. This has the advantage that it is also possible to compensate for changes or inaccuracies that are provoked subsequently during the therapeutic and/or diagnostic measure. This provides that the method is also executed as an iterative method, and may therefore achieve a particularly high level of accuracy.

In one embodiment, provision is made for registering the initial image data record with the intraoperative image data record as a further act before comparing the position and/or the orientation of the end effector with the landmark. An association is thus established between the landmark of the initial image data record and the intraoperative image data record. This need not be performed separately if the initial image data record and the intraoperative image data record are created using the same device, for example, since a shared system of coordinates is then available for both image data records. This has the advantage that the initial image data record and the intraoperative image data record need not be created using the same imaging system. This allows for a reduction in any effective radiation exposure and greater flexibility in the execution of operations.

In a particularly advantageous embodiment, provision is made for performing the cited act for all of the landmarks if more than one landmark is selected. This has the advantage that, if the landmarks are used for a series of therapeutic and/or diagnostic measures, it is possible to compensate for a deterioration in registration accuracy that is caused by one of the therapeutic and/or diagnostic measures at one of the landmarks.

In a further embodiment, provision is made for the medical robotic device autonomously to move the end effector and/or autonomously to influence its movability by an operator. The medical robotic device may therefore autonomously initiate a movement of the end effector and/or restrict the degree of freedom of the end effector, such that, e.g., in a so-called “gravity mode” by virtue of so-called “active constraints” the end effector may then only be moved, by pressing or pushing by an operator, in a direction that is determined by the medical robotic device. This has the advantage that the medical robotic device may reposition itself automatically and may therefore compensate for the deterioration in registration accuracy very precisely. In the case of a movement carried out by an operator and is controlled by the medical robotic device, the high level of accuracy of the robotic guidance is combined with the human attentiveness and the corresponding direct feedback to the operator.

In a particularly advantageous embodiment, provision is made for performing acts d) to g) if a measure for the inaccuracy of the positioning in act c), e.g., a measure for poor registration accuracy at the time of the positioning in act c), exceeds a predefined limit value. In particular, acts d) to g) may then be performed automatically. This has the advantage that time is saved during the operative and/or diagnostic intervention, since the compensation only takes place when necessary, and furthermore if an x-ray recording is carried out for the intraoperative image data record, e.g. radiation exposure is reduced in respect of the body to be operated on. At the same time, the advantage of the increased accuracy is preserved.

In this case, the measure may in particular take into consideration a time that has elapsed since the previous recording of an intraoperative image data record. This has the advantage that it is possible in a simple manner to compensate for a deterioration in the registration accuracy when the deterioration may increase over an elapsed time.

Alternatively or additionally in this case, the measure may take into consideration knowledge of movements previously performed by the medical robotic device, and any kinematic inaccuracy of the medical robotic device resulting from the movements. For example, previously performed movements may be evaluated here using an absolute measure for the kinematic inaccuracy. Therefore, if the medical robotic device has performed a series of movements that are known to be associated with a higher kinematic inaccuracy of the medical robotic device than other movements, for example, this may be taken into consideration to the effect that the compensation takes place earlier than in the case of movements that are associated with only slight inaccuracies. This has the advantage of a specifically configured compensation and correspondingly minimal radiation exposure, for example.

In a further embodiment, the initial image data record represents a three-dimensional image and at least one intraoperative image data record represents only a two-dimensional image. In particular, this may be a three-dimensional image that has high resolution in comparison with the two-dimensional image. This has the advantage that the intraoperative image data record may be registered with the initial image data record, thereby allowing the position of the end effector to be determined relative to the landmarks, and specifically with good accuracy and at low cost. Only modest radiation exposure is incurred, since less radiation exposure occurs for a two-dimensional image than in the case of a three-dimensional image.

In a further embodiment, the medical robotic device has a biopsy needle and/or a drilling template as an end effector. This has the advantage that greater accuracy may be achieved even if, e.g., a soft tissue organ moves during partially or fully automated biopsy extraction, and/or greater accuracy may be achieved in respect of the drilling template for pedicle screws, for example that are inserted. A particularly high level of precision is particularly important in precisely these two areas of application.

In a further embodiment, the body to be operated on is a human patient, particularly a spinal column of a human patient.

### BRIEF DESCRIPTION OF THE DRAWING

FIG. 1 depicts a schematic representation of an example of a spinal column and a medical robotic device.

### DETAILED DESCRIPTION

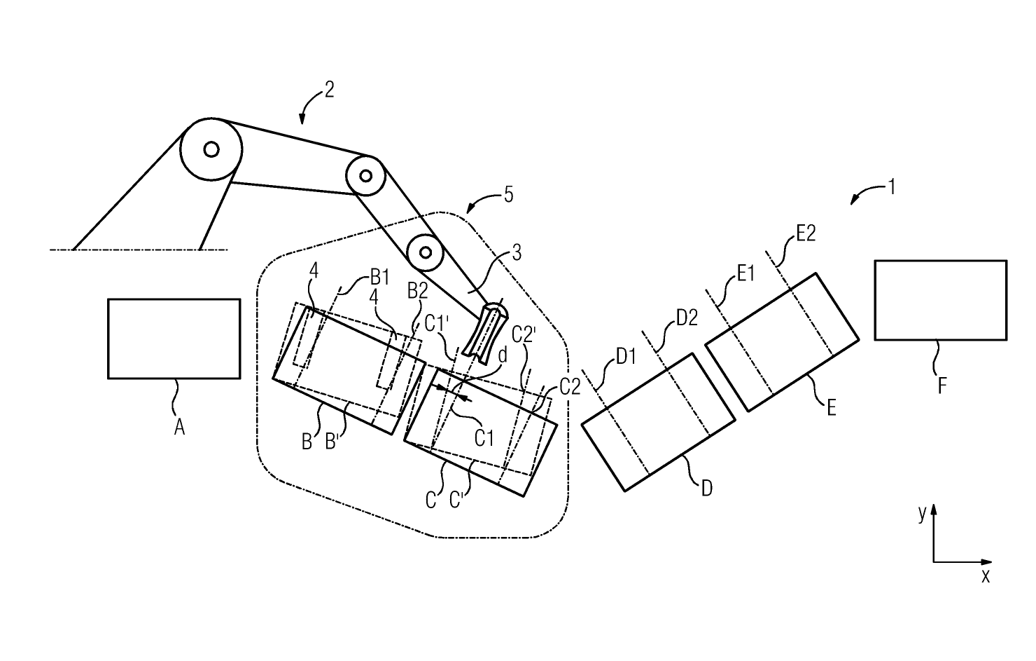

In FIG. 1, six vertebrae A to F are represented as unbroken rectangles. The vertebrae are arranged adjacent to each other in a curved line. In the example depicted, the curved line is to be corrected by an operative intervention. To this end, respective so-called pedicle screws are screwed into the vertebrae B to E, serving then to pull the vertebrae into a position that differs from the present position and thereby promoting a recovery process. Respective landmarks B1, B2, C1, C2, D1, D2, E2, E1 (B1-E1) here determine the positions at which respective pedicle screws are to be screwed into the vertebrae B to E. To this end, a hole is first drilled along the landmarks B1 to E2 in FIG. 1, such that a pedicle screw may then be screwed into each hole. This drilling takes place semi-automatically in the example depicted. For example, a medical robotic device 2 having an end effector 3 embodied as a drilling bush is positioned at the respective landmarks B1 to E1 in such a way that an operator, guided by the drilling bush, may precisely drill the intended holes in each case.

In FIG. 1, two holes 4 have already been drilled in the vertebra B. These correspond exactly to the landmarks B1, B2 of the vertebra B. Registration of the medical robotic device 2 relative to the body to be operated on 1, the spinal column here, was performed before making the drilled holes in the vertebra B in this case, such that the end effector 3 here is in a well-defined position relative to the body to be operated on, e.g., the landmarks B1 to E2. However, in FIG. 1, possibly as a result of drilling the holes 4, the vertebra B and the vertebra C have moved into the new positions B′ and C′ respectively. Consequently, the landmarks C1, C2 of the vertebra C, which are based on the original position of the vertebra C, no longer correspond to the correct drilled holes. If drilling was actually performed according to the original landmarks C1, C2 in this situation, resource-intensive corrections may subsequently be required.

According to the method, during the operative intervention, a landmark C1, C2 of the vertebra into which a hole will next be drilled is now selected, e.g., the landmark C1. The end effector 3 is positioned in the vicinity of the landmark C1. In this case, a recording is now made of an intraoperative image data record, an area 5 here, which covers a region of the body 1 adjacent to the end effector 3 and includes landmarks B1, B2, C1, C2 in the drawing. By registering the initial image data record, e.g., the vertebrae A to F represented by the unbroken rectangles here, with the intraoperative image data record, e.g., the unchanged vertebrae A, D, E, F and the vertebrae B, C having the new positions B′, C′, the selected landmark C1 is associated with the intraoperative image data record. This results in the new position C1′ of the landmark C1, which is moved and rotated in this case by a change d relative to the original position.

A determination of the position and/or orientation of the end effector 3 in the intraoperative image data record now depicts that the end effector 3 is still directed at the original position of the landmark C1. If, in a further act, the actual position of the end effector 3 is now compared with the new position C1′ of the landmark C1, the divergence d is then identified. By repositioning the end effector 3 to a new position, which corresponds to the new position C1′ of the landmark C1, this divergence d is now compensated for. Consequently, in FIG. 1, the drilled hole for the pedicle screw is precisely guided relative to the vertebra C by the drilling template, and is unaffected by the change in the anatomy and/or geometry of the body to be operated on, the change having taken place during the intervention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for compensating for a deterioration in registration accuracy of a medical robotic device relative to a body to be operated on, the deterioration occurring during an operative intervention, a diagnostic intervention, or an operative and diagnostic intervention, by repositioning the medical robotic device comprising an end effector for performing a diagnostic measure, a therapeutic measure, or both diagnostic and therapeutic measures, the method comprising:
selecting at least one landmark, which is to be reached by the end effector, on an initial image data record of the body to be operated on;
registering the medical robotic device relative to the body;
positioning the end effector in the vicinity of the landmark;
recording an intraoperative image data record in which the end effector is captured with a region of the body that is adjacent to the end effector, the region comprising a section of the body featuring the landmark in the initial image data record;
determining a position, an orientation, or the position and the orientation of the end effector in the intraoperative image data record;
comparing the position, the orientation, or the position and the orientation with the landmark and identifying any divergence present; and
repositioning the end effector in order to compensate for the divergence.

2. The method as claimed in claim 1, wherein the recording, the determining, the comparing, and the repositioning are performed repeatedly during the operative intervention, the diagnostic intervention, or the operative and diagnostic intervention.

3. The method as claimed in claim 2, wherein the initial image data record is a preoperative image data record, and wherein a registration of the initial image data record with the intraoperative image data record is performed, thereby associating the landmark of the initial image data record with the intraoperative image data record before the position, the orientation, or the position and the orientation of the end effector is compared with the landmark.

4. The method as claimed in claim 2, wherein the medical robotic device autonomously moves the end effector, autonomously influences a movability of the end effector by an operator, or autonomously moves the end effector and autonomously influences the movability of the end effector by the operator.

5. The method as claimed in claim 2, wherein the initial image data record represents a three-dimensional image and the intraoperative image data record represents a two-dimensional image.

6. The method as claimed in claim 1, wherein the initial image data record is a preoperative image data record, and wherein a registration of the initial image data record with the intraoperative image data record is performed, thereby associating the landmark of the initial image data record with the intraoperative image data record before the position, the orientation, or the position and the orientation of the end effector is compared with the landmark.

7. The method as claimed in claim 6, wherein the medical robotic device autonomously moves the end effector, autonomously influences a movability of the end effector by an operator, or autonomously moves the end effector and autonomously influences the movability of the end effector by the operator.

8. The method as claimed in claim 6, wherein the recording, the determining, the comparing, and the repositioning are performed when a measure for the inaccuracy of the positioning in the positioning of the end effector exceeds a predefined limit value.

9. The method as claimed in claim 6, wherein the initial image data record represents a three-dimensional image and the intraoperative image data record represents a two-dimensional image.

10. The method as claimed in claim 1, wherein the medical robotic device autonomously moves the end effector, autonomously influences a movability of the end effector by an operator, or autonomously moves the end effector and autonomously influences the movability of the end effector by the operator.

11. The method as claimed in claim 10, wherein the recording, the determining, the comparing, and the repositioning are performed when a measure for the inaccuracy of the positioning in the positioning of the end effector exceeds a predefined limit value.

12. The method as claimed in claim 10, wherein the initial image data record represents a three-dimensional image and the intraoperative image data record represents a two-dimensional image.

13. The method as claimed in claim 1, wherein the recording, the determining, the comparing, and the repositioning are performed when a measure for the inaccuracy of the positioning in the positioning of the end effector exceeds a predefined limit value.

14. The method as claimed in claim 13, wherein the measure takes into consideration a time that has elapsed since a previous recording of an intraoperative image data record.

15. The method as claimed in claim 14, wherein the measure further takes into consideration knowledge of movements performed by the medical robotic device and a kinematic inaccuracy of the medical robotic device resulting from the movements.

16. The method as claimed in claim 13, wherein the measure takes into consideration knowledge of movements performed by the medical robotic device and a kinematic inaccuracy of the medical robotic device resulting from the movements.

17. The method as claimed in claim 1, wherein the initial image data record represents a three-dimensional image and the intraoperative image data record represents a two-dimensional image.

18. The method as claimed in claim 1, wherein the end effector is a biopsy needle, a drilling template, or the biopsy needle and the drilling template.

19. The method as claimed in claim 1, wherein the body to be operated on is a human patient.

20. The method as claimed in claim 1, wherein the body to be operated on is a spinal column of a human patient.
